(19) United States
(12) Patent Application Publication
(10) Pub. No.: US 2009/0018451 A1
(43) Pub. Date: Jan. 15, 2009

(54) Dynamic Sampling System and Method for In Vivo Fluorescent Molecular Imaging

(76) Inventors: Jing Bai, HaiDian District (CN); Xiaolei Song, HaiDian District (CN); Gang Hu, HaiDian District (CN); Junjie Yao, HaiDian District (CN); Zhun Xu, HaiDian District (CN); Daifa Wang, HaiDian District (CN); Jianchang Ou, HaiDian District (CN); Guoliang Huang, HaiDian District (CN); Yonghong Zhang, HaiDian District (CN)

Correspondence Address: HAYNES AND BOONE, LLP;IP Section, 2323 Victory Avenue, Suite 700, Dallas, TX, 75219, US

(21) Appl. No.: 12/053,355

(22) Filed: Mar. 21, 2008

Related U.S. Application Data
(63) Continuation of application No. PCT/CN2007/002114, filed on Jul. 10, 2007.

(51) Int. Cl. A61B 5/00

(52) U.S. Cl. 600/476

(57) ABSTRACT

A dynamic dada sampling system and method is disclosed for in vivo small animal fluorescence molecular imaging and dual-modality molecular imaging. The system comprises a computer, a rotation stage for animal suspension driven by a motor, and a fluorescence excitation-detection apparatus. The fluorescence excitation-detection apparatus comprises a fluorescence excitation module and a fluorescence detection module. The CCD device of the fluorescence detection module is connected to a computer through an interface controller. The motor is connected to a computer through RS232 interface. The process of dynamic data acquisition is as follows: a fluorescent probe is injected into a small animal in order to target specific cells or tissues; a small animal is vertically hung on a rotation stage after anesthesia; the fluorescence imaging detection module acquires the emitting light continuously. The present invention can provide 360 degree imaging quickly, efficiently, and non-invasively.

### CROSS REFERENCE TO RELATED APPLICATIONS

This application is a continuation of PCT/CN2007/002114, filed Jul. 10, 2007, now pending, the entire contents of which are hereby incorporated herein by express reference thereto.

### FIELD OF THE INVENTION

The present invention relates to an imaging system and method. In particular, the present invention relates to an imaging system and a method used in dynamic sampling imaging data while a small animal rotates.

### BACKGROUND OF THE INVENTION

Medical imaging has experienced a revolution from a discipline based on anatomy to one that is increasingly based on tissue function. Molecular imaging is a relatively new technology, which is defined as measurements and/or images of specific molecules (genes, proteins, etc.) and molecular pathways in vivo through the injection of a probe which is chosen to target an intrinsic molecule in the living specimen or enables the visualization of the molecule of interest. It is a technique by which one can obtain data about physiological processes at the molecular level. It allows us to detect diseases much earlier, to stage cancer and some other diseases much more accurately and noninvasively, and to facilitate and speed up the drug development process. There are several imaging techniques in molecular imaging, such as PET, MRI, and optical imaging. Optical molecular imaging based on fluorescent probes has emerged as the most promising new technique, with the advantages of noninvasive measurement, low cost facilities, a wealth of fluorescent probes, and a matured technique of probe target interaction. Photon transport in biological tissue is severely affected by the processes of absorption and scattering. Because of low tissue absorption in the near-infrared window, the light with the wavelength in the near-infrared range can propagate through tissue for distances in the order of several centimeters. Currently, optical imaging has been extensively used in investigation of small animals in vivo. It is a straightforward translation from in vitro experiment to the clinical application.

Optical imaging techniques can be classified as planar and tomographic approaches. In planar imaging, light is shined through the tissue when the source and the detector are placed on the opposite sides of the tissue, and the relative attenuation of fluorescent light emitted is recorded. The operational principle of tomography resembles that of X-ray computed tomography (CT). The tissue is illuminated at different points or projections and the collected light is used in combination with a mathematical formulation that describes photon propagation in tissue. While 2D image of a projection is available in planar imaging, 3D fluorochrome concentration distribution can be mathematically reconstructed using the light propagating through tissues by combining data acquired at different projections.

Optical molecular imaging includes bioluminescence tomography (BLT) and fluorescence molecular tomography (FMT). BLT is used to localize and quantify bioluminescent sources in a small living animal. FMT is performed by irradiating the tissue with a frequency of light lower than the emission-frequency-exciting fluorescence from intrinsic or extrinsic probes under investigation. FMT offers distinct advantages over BLT as fluorescence probes generate a much stronger signal than bioluminescence probes and yield a higher signal-to-noise ratio.

Several FMT systems have been developed during the last decade. The initial system is a cylindrical FMT system for mouse imaging. Excitation and collection fibers are arranged around an optical bore to deliver and collect light. The tube is filled with a matching fluid, whose optical properties are similar to those of human tissue, to minimize photon wave mismatches within the cylinder. However, the implementation has the inherent disadvantage of the sparse spatial sampling due to the limited detection channels, which could deteriorate the imaging resolution. Later, a FMT system consisting of a parallel plate-imaging chamber and a lens coupled CCD camera has been developed, which enables conventional planar imaging with a spatial resolution of sub-millimeter. However, the system still has compromised resolution along the axis perpendicular to the detector plane due to the limited projection angles employed. Recently, a free-space FMT imager utilizing 360 degree geometry projections has been developed for non-contact detection and illumination, which shows powerful abilities to obtain complete projection and symmetrical scan by rotating the equipment or rotating the animal body. These imagers offer high-quality data sets due to the high spatial sampling of photon fields allowed at any projection, and significant experimental simplicity since no matching fluids are utilized and no fibers are brought in contact with tissue. However, the measurement of light intensity is accomplished in a step by step acquisition format, which is time-consuming due to the alternate hardware control of mechanical rotation and photon collection. Furthermore, the step by step acquisition format may cause unwanted soft tissue movements during living animal experiments when the scan rotation starts and stops.

Multimodality imaging approaches is a very attractive strategy. Coregistration with other modalities can be helpful in understanding the source of contrast. For example, X-ray CT or PET can yield even more findings by colocalizing anatomical and molecular contrast. A dual-modality PET/CT brings together PET's sensitivity in assessing physiological and metabolic states with CT's precise anatomical information. It gives invaluable information for cancer diagnose and treatment in the clinic.

A dual-modality system combines high-sensitivity optical molecular imaging with high-resolution digital X-ray to enable quantification and localization of biomarkers in small animal imaging. The acquisition method usually is in a consecutive mode, data sampling first from one modality, and then from the other. This method does not permit simultaneous multi-modality imaging.

A new data acquisition method of FMT is needed to acquire the fluorescent light quickly, to integrate with other imaging modalities easily, and to realize multi-modality imaging simultaneously.

In view of the foregoing, in one embodiment the present invention provides a dynamic data sampling method that enables the capture of the light emitted from the boundary of the animal while it is being rotated.

In another embodiment the present invention provides a dynamic data sampling system that enables the capture of the light emitted from the boundary of the animal while it is being rotated.

In another embodiment, the present invention provides a dynamic data sampling method that enables to capture of the light emitted from the boundary of the animal that is being rotated continuously at an appropriate speed throughout the measurement cycle.

In yet a further embodiment, the present invention provides a dynamic data sampling system that enables to capture of the light emitted from the boundary of the animal that is being rotated continuously at an appropriate speed throughout the measurement cycle.

### SUMMARY OF THE INVENTION

To obtain the above-noted embodiments, separately or in any combination, the present invention provides a system and a method for dynamically obtaining raw image data that represent fluorescent light distribution inside an animal or a biological sample. The distribution may indicate the location, size and concentration of one or more fluorescent probes in the animal.

The system of the present invention comprises a computer, a rotation stage configured to support the animal or the biological sample and a fluorescence excitation-detection apparatus. The rotation stage includes a device for animal suspension and at least one motor for driving the device. The fluorescence excitation-detection apparatus comprises a fluorescence excitation module and a fluorescence detection module. The fluorescence excitation module further comprises a fluorescence excitation source, a focalizing lens, and scan control unit. The fluorescence detection module comprises a band-pass filter and a CCD device, which is connected to a computer through an interface controller. The motor is connected to a computer through RS232 interface. The input of the computer comes from the output of CCD device. The components controlled by the computer include the CCD device and the at least one motor.

Preferably, the at least one motor comprises two stepper motors, one for driving the stage to rotate around its vertical axis, and the other for lifting or lowering the stage along its vertical axis.

An animal contour acquisition device is set at the same side of the fluorescence excitation module.

The complete 360 degree space around the animal suspension stage could be divided into four equal areas for dual-modality imaging. Each pair of two opposite areas is applied for one imaging modality. For example, fluorescence imaging modality employs a pair of two areas where the excitation and detection modules located on the opposite sides of the stage. A piece of leaded glass which is shielded against high energy photons is placed in front of the fluorescence detection module. PET imaging modality employs the other pair of two areas where the PET detectors are located. In this case, a dual-modality system of FMT/PET is available. When the PET imaging system is replaced by X-ray CT imaging system, X-ray emission and detection modules are located on the opposite sides of the stage, a dual-modality system of FMT/CT is available too. The complete 360 degree space around the stage could be divided into six equal areas; each of them is a 60 degree section. When a fluorescence excitation module and a detection module locates on a pair of opposite two areas, PET detectors locate on the second pair of opposite two areas, and X-ray emission and detection apparatuses locate on the third pair of opposite two areas, a multi-modality system of FMT/PET/CT is available for in vivo imaging of a small animal.

A dynamic data sampling imaging method comprises following steps. A fluorescent probe that labels a specific cell or tissue is introduced into a small living animal by injection. The animal is vertically hung on the stage after anaesthesia. During the scan, the animal stage rotates continuously around its vertical axis. The rotation in a uniform speed is controlled by a stepper motor. At the same time, the fluorescence detection module collects photons propagating from the surface of the animal and sends the raw imaging data to the computer. After a 360 degree rotation, the animal stage is lifted along its vertical axis by another stepper motor in order to scan another part of the body, which depends on the detection field of view and other imaging requirements.

Coupled with the rotational stage, the fluorescence excitation-detection apparatus is characteristic of non-contact photon delivery and 360 degree full view detection. The process of data acquisition is as follows. The excitation light of the fluorescence excitation module is focused on the surface of the animal via a lens. The light enters the animal's body, interacts with the molecular probes inside, which in turn emit fluorescence. The fluorescence photons travel inside the animal body, emit out from the surface of the animal body, and then are captured by the fluorescence detection module. In the fluorescence detection module, photons first pass a band-pass filter, and then are collected by the CCD device whose output is connected to the computer.

Because the intensity of the fluorescence emitted from the surface of animal is usually weak, it is common that an exposure time of several seconds is needed to get an image with acceptable signal noise ratio. However, since the stage rotates continuously, the excitation light spot laid on the animal surface changes during the exposure time, as well as the field view of the CCD device. So the raw image data obtained should be recalibrated as follows before they are used for image reconstruction. The calibrated excitation light spot for the acquired image is set as the center point of the curve that traces the light spots on the animal surface during the exposure time. The field of view of the CCD device covers an approximately 120° angular section from the rotation axis point. For the raw image data, those outside the 90 degree field of view from the rotation axis point are discarded. Data inside of the 90 degree field of view are reserved for image reconstruction. After the process of approximation and calibration, reconstruction algorithms developed for static data acquisition can be employed herein.

The present invention with the above technical scheme has several features as follows.

(1) By using a uniform-speed rotation scheme, the dynamic data sampling method can acquire the 360 degree imaging data quickly and efficiently, thus provide a high throughput imaging technology. (2) The method also provides a more stable imaging technique. As the animal is in a relative stable state under the rotation at a slow uniform speed, the method eliminates organ movements in a step by step rotation. (3) Furthermore, motor control technique of the uniform-speed rotation is more simple compared to an angular step rotation. (4) The 360 degree full view dynamic data acquisition method offers high spatial sampling of photon fields and significant experimental simplicity. (5) When the invention integrates with other imaging modality, such as PET and/or CT, a dual-modality and/or multi-modality imaging system can be obtained that provides functional and/or molecular information as well as precise structural information.

### BRIEF DESCRIPTION OF THE DRAWINGS

Further features and advantages of the invention can be ascertained from the following detailed description that is provided in connection with the drawing(s) in the accompanying figures:

FIG. 1 illustrates the three components of the dynamic data sampling method for in vivo fluorescence molecular imaging of a small animal.

FIG. 2 is the schematic diagram of the components of the imaging system.

FIG. 3 is the schematic diagram for imaging data acquisition process.

FIG. 4 shows the spectrum characteristics of a fluorescent probe Cy5.5 and XF3113 (710AF40) filter. The thick solid line indicates the penetration rate of the XF3113, the dotted line indicates the optical excitation spectra of the Cy5.5, and the thin line indicates the fluorescence spectroscopy of the Cy5.5.

FIG. 5 illustrates the recalibration method of the dynamic imaging data sampling.

FIG. 6 is a top view of the experimental setup for fluorescence measurements.

FIG. 7 shows transillumination phantom fluorescence images: (a) is a still image at reference 0° position with fluorescence probe at depth of 2.5 mm; (b)˜(d) are dynamic acquisition images at rotation speeds of 0.6°/s, 1.2°/s and 2.4°/s; (e)˜(g) are the normalized images created by dividing (b)˜(d) with (a), respectively; (h)˜(n) are the images at 180° position with fluorescence probe at depth of 22.5 mm with similar treatments as (a)˜(g).

FIG. 8 shows tomographic slices reconstructed from dynamic data set obtained at rotation speeds of (a) 0.6°/s, (b) 1.2°/s and (c) 2.4°/s.

FIG. 9 illustrates a schematic diagram of the imaging system with an animal contour acquisition device placed at the same side of the fluorescence excitation module.

FIG. 10 is a schematic diagram for imaging data acquisition process with small animal contour acquisition.

FIG. 11 illustrates the spatial arrangement of PET/FMT dual-modality system

FIG. 12 illustrates the components of PET/FMT dual-modality system

FIG. 13 is a schematic diagram of imaging data acquisition process in PET/FMT dual-modality system

### DETAILED DESCRIPTION OF THE PREFERRED EMBODIMENTS

In the following detailed description of the present invention, several specific embodiments are set forth in order to provide a thorough understanding of the invention.

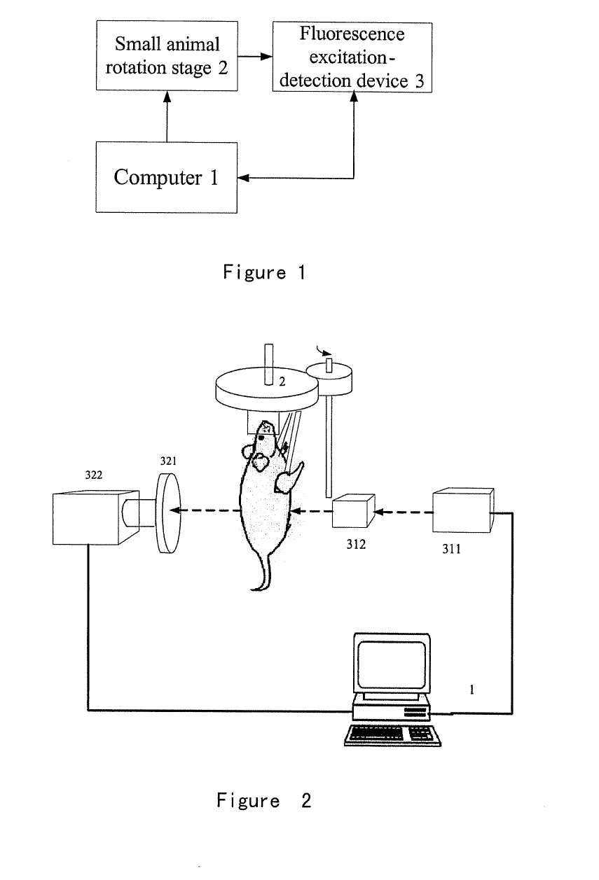

As shown in FIG. 1, this invention includes a computer 1, a small animal rotation stage 2, and a fluorescence imaging excitation-detection device 3. The rotation stage 2 includes a device for animal suspension and one or more stepper motors. The fluorescence imaging excitation-detection devices 3 includes fluorescence excitation module 31 and fluorescence imaging detection module 32. Fluorescence excitation module 31 includes excitation light source 311 and focusing and scanning control unit 312. Fluorescence imaging detection module 32 includes a band-pass filter 321 and a CCD device 323 which comprises a lens and a CCD camera. An excitation light source 311 provides the initial energy for fluorescent probe, which could be a combination of a broad spectrum incandescent bulb and different band-pass filters or a narrow band laser with a specific central wavelength. In the focusing and scanning control unit 312, an excitation laser beam is focused to a small light spot illuminating the skin surface of the small animal, and the movement of the light spot along the surface of the small animal is controlled by the scan control unit. In the fluorescence imaging detection module 32, the band-pass filter 321 rejects the excitation light and other light noise. The objective lens of the CCD devices 322 has a large numerical aperture. With an external water-cooling circulation, the CCD camera is cooled down to −85° C. to reduce the dark current noise. Interactive communication between the computer and the CCD device is realized by an interface controller. The output of CCD device is sent to the computer for image reconstruction. The small animal rotation stage is driven by the two stepper motors, which is connected to the computer 1 through the RS232 interface and controlled by computer software. One of the stepper motors is employed to drive the stage to rotate around its vertical axis in a step or uniform mode by setting a rotation angle and speed, and the other is employed to lift and lower the stage along its vertical axis. The CCD device 322 output the raw data for image reconstruction to the input of the computer 1. The output of the computer controls the CCD device 322 and the two stepper motors.

In FIG. 2, the small animal rotation stage 2 is the key component of the invention. The small animal is suspended vertically with its upper limbs fixed to the rotation stage 2. The anterior-posterior axis of the animal is set to coincide with the axis of the rotation stage 2. Fluorescence imaging excitation module and detection module are placed on the opposite sides of the rotation stage 2. During the 360 degree fall view scan of small animal fluorescence images, the animal stage 2 controlled by a stepper motor, rotates uniformly around its vertical axis at a particular speed. At the same time, the CCD device continuously acquires raw data for medical imaging. After a 360 degree rotation, the animal stage is lifted along its vertical axis by another stepper motor in order to scan another part of the body, depending on the detection field of view and other imaging requirements.

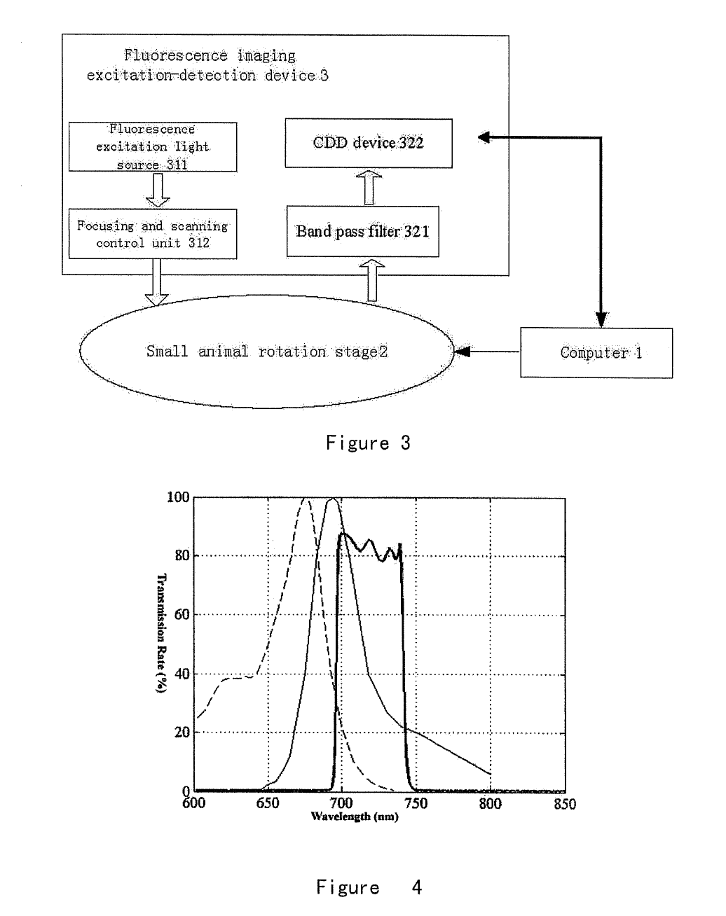

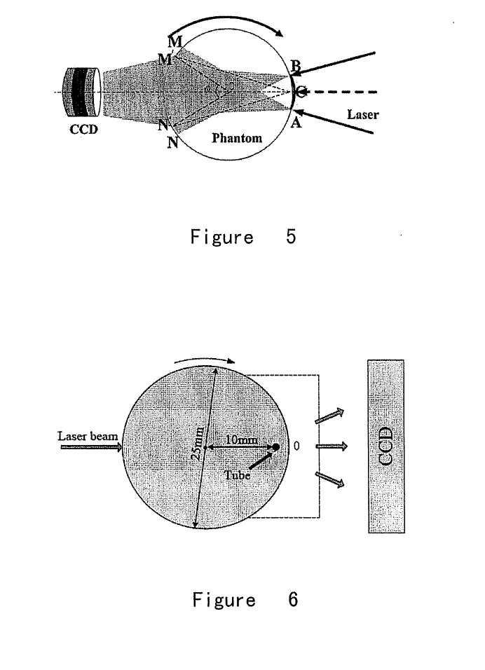

In FIG. 3, the wide arrows indicate optical signal flows and the thick solid line arrows indicate electric signal flows. A fluorescent light with a longer wavelength than the excitation light is generated by irradiation. It travels through the small animal tissue and becomes very weak when it is detected by an optoelectronic device. The fluorescence imaging excitation-detection device 3 is a non-contact, 360-degree full view detection system. Here the excitation light source 311 of the fluorescence imaging laser module 31 is a laser beam. The excitation light is focused on the surface of the animal via a lens of the focusing and scanning control unit 312, and then enters the animal's body. The fluorescent probe inside the animal, targeting specific tissues or cells, is excited and emits fluorescence. The fluorescence photons travel inside the animal body, emit out from the surface of the animal body, and then are captured by the fluorescence detection module 32. In the fluorescence detection module, photons first pass a band-pass filter 321 to eliminate the excitation light and the other light noise, and then are collected by the CCD device 322 whose output is sent to the computer for image reconstruction and 3D fluorochrome distribution location. In this embodiment, the fluorescent probe is a Cy5.5, band-pass filter is a XF3113 (710AF40) commercially available from the United States OMEGA OPTICAL Company. FIG. 4 shows the spectral characteristics of fluorescent probe Cy5.5 and XF3113 filters. The thick solid line indicates the penetration rate of XF3113, the dotted line indicates optical excitation spectra of Cy5.5, and the thin solid line indicates fluorescence spectroscopy of Cy5.5. The CCD camera is a DU-897 Electron Multiplying CCD commercially available from the British Andor Company.

The operation steps of the present invention are as follows:

1. Marker preparation: a fluorescent probe targeting specific cells or tissues is introduced into a small animal by injection.

2. Animal fixation: upper limbs of the small animal are fixed on the rotation stage after anesthesia.

3. Data acquisition: controlled by a stepper motor, the animal stage rotates continuously around its vertical axis at a slow speed. During the scan, the excitation light of the fluorescence excitation module illuminates on the surface of the animal continuously, and the photons emitting from the fluorescent probe are continuously captured by the fluorescence detection module at same time. The imaging raw data is sent to the computer.

4. After a 360 degree rotation, the animal stage is lifted along its vertical axis by another stepper motor in order to scan another part of the body, depending on the detection field of view and other imaging requirements.

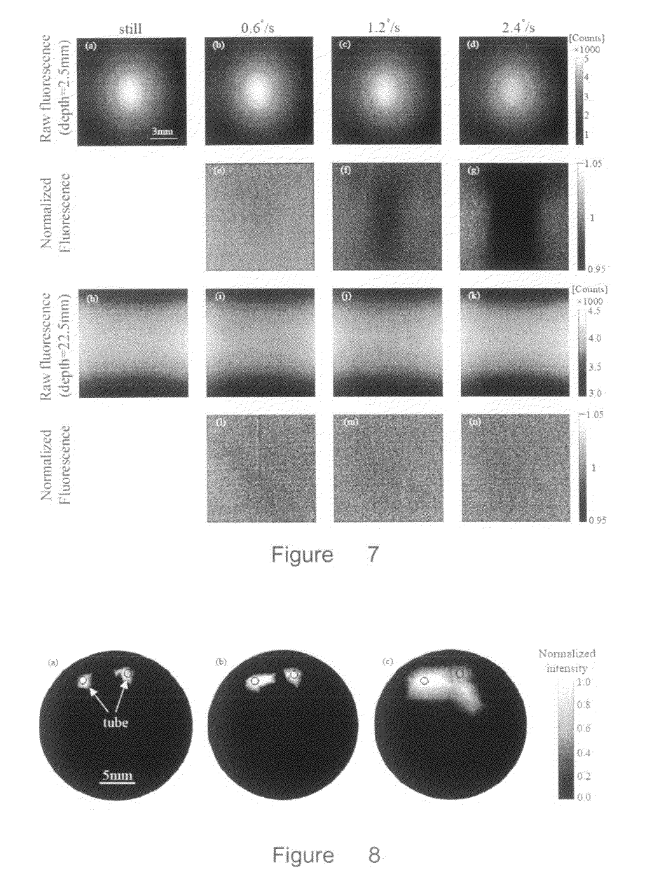

Because the intensity of the fluorescence emitted from the surface of animal is usually weak, it is common that several seconds exposure time is needed to get an image with an acceptable signal noise ratio. However, since the stage rotates continuously, the excitation light spot laid on the animal surface changes during the exposure time, as well as the field view of the CCD device, as illustrated in FIG. 5. During the exposure time, the excitation light spot moves from A to B on the animal surface, and the field of view of the CCD device covers the area from M to N. The field of view of the CCD device covers an angular section of φ degree from the rotation axis point, herein approximately 120°. So the raw image data obtained should be recalibrated as follows before they are used for image reconstruction. After the process of approximation and calibration, reconstruction algorithms developed for static data acquisition can be employed herein.
(1) The calibrated excitation light spot for the acquired image is set as the center point of the curve that traces the light spots on the animal surface during the exposure time. In FIG. 5, the calibrated excitation light spot is C dot pointed by a dashed arrow, and the field of view of the CCD device is the area of M′ N′.
(2) For the raw image data, those outside the 90 degree field of view from the rotation axis point are discarded. Data inside the 90 degree field of view are reserved for image reconstruction.

In order to verify the above dynamic data acquisition method, a series phantom experiments have been done. FIG. 6 is the top view of the experimental arrangement. A 25 mm-inner-diameter glass container filled with turbid medium (μₐ=0.25 cm⁻¹, μ′ₛ=10 cm⁻¹) simulates the optical properties of small animals, which is fixed vertically on a rotation stage (Huawei Haorun, China). A 0.7 mm-inner-diameter, 6mm-length glass capillary infused with 4 μM concentration Cy5.5 dye acts as the fluorescent target, which is immersed into the diffuse solution container and fixed at a 10 mm distance to the center axis of rotation. The stage can be configured in a step or continuous rotation mode with different speed to realize a 360° scan of the target. All operations of mechanical rotation and image capture are controlled by a computer.

FIG. 7 is transillumination fluorescence images of the phantom. FIG. 6(a) is a still image at reference position of 0°, with fluorescence probe of 2.5 mm-depth to the CCD focal plane. FIG. 6(b)˜(d) are dynamic acquisition images with rotation speed of 0.6°/s, 1.2°/s and 2.4°/s. FIG. 6(e)˜(g) are the normalized images created by dividing (b)˜(d) with (a), respectively. Similarly, FIG. 6(h) is a still image at reference position of 180°, with fluorescence probe of 22.5 mm-depth to the CCD focal plane. FIG. 6 (i)˜(k) are dynamic acquisition images with rotation speed of 0.6°/s, 1.2°/s and 2.4°/s. FIG. 6(l)˜(n) depict the corresponding fluorescence dynamic acquisition images normalized to the reference images (h). The normalized fluorescence dynamic images acquired from the two slow speed of 0.6°/s and 1.2°/s tests do not show significant variations in the distribution field wherever the fluorescent tube is located.

FIG. 8 are tomographic measurement experiments of phantom container in FIG. 6. Two glass capillaries with the same size in FIG. 6, both filled with 2 μM concentration Cy5.5 dye, are embedded into the turbid solution that has the same recipe as in the above experiments. The distance between the two capillaries is 6 mm. The dynamic datasets of fluorescent signals are obtained in trans-illumination mode with rotation speed of 0.6°/s, 1.2°/s and 2.4°/s. The exposure time is fixed at 5 seconds to generate 120, 60 and 30 data frames in 360 measurement. In each frame, a 41×41 virtual detector array in the center region on the phantom surface is extracted for inversion reconstruction. The dynamic frame images are substituted from the still images collected at the middle angular position of the covering region caused by rotation. The reconstructed transversal slices are shown in FIG. 8. The black circles denote the real position of the two tubes. It can be seen that the tomographic images based on dynamic data sets from slow speed experiments can locate the position of the fluorophores and report an approximate concentration ratio.

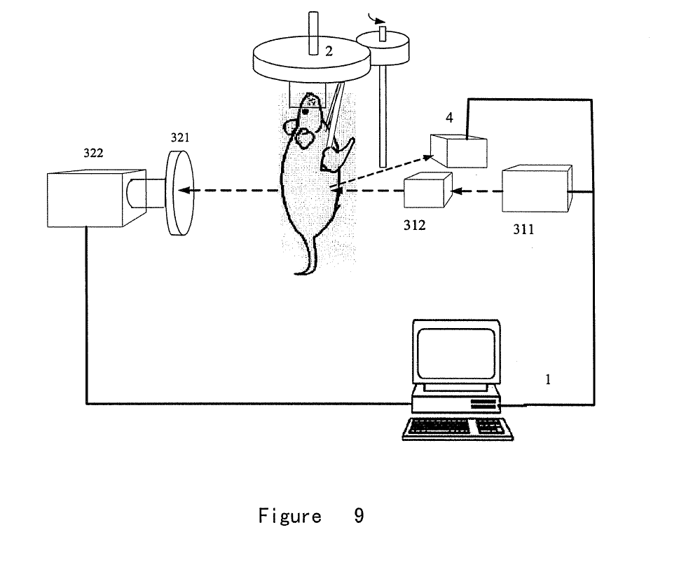

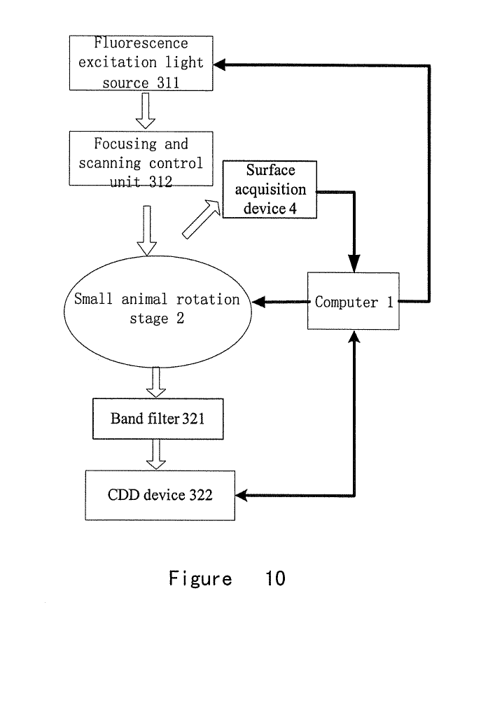

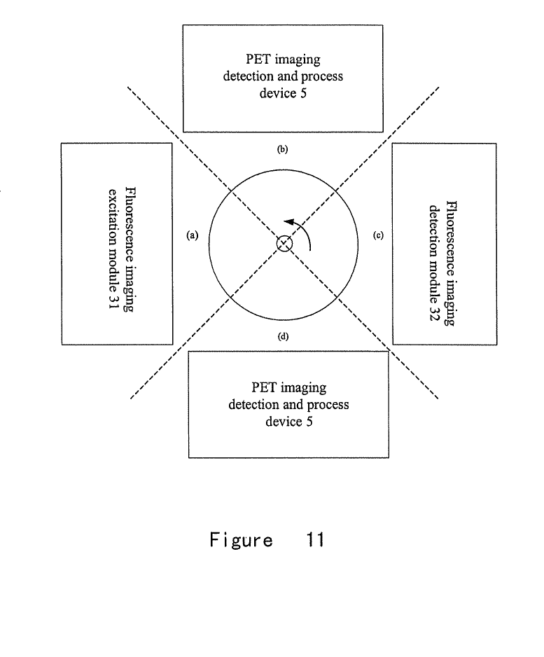

Based on the above excitation fluorescence tomography of FIG. 2, an animal contour acquisition device 4 is added in FIG. 9. The animal contour acquisition device 4 is placed at the same side of the fluorescence excitation module 31. In the process of fluorescence images acquisition, the 3D surface contour information is acquired at the same time. The back-projection method can be used to reconstruct 3D surface contour of the small animal from a group of photos. FIG. 10 is a schematic diagram for imaging data acquisition process with small animal contour acquisition. In this implementation, the animal contour acquisition device 4 is a camera connected to the computer.

The present invention can also be integrated with other imaging devices by a proper spatial arrangement, and a dual-modality and/or multi-modality imaging system are available to provide different information of the small animal.

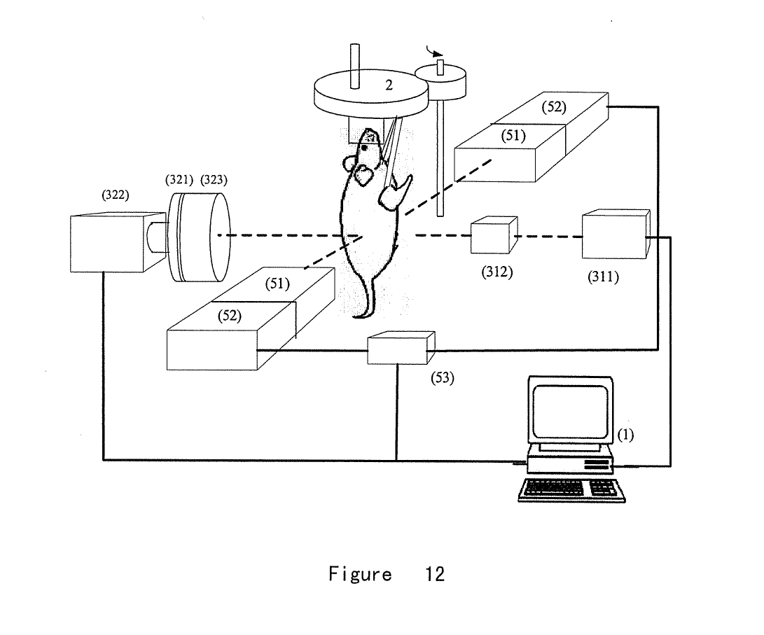

As shown in FIG. 11, based on the above excitation fluorescence tomography of FIG. 2, when adding a PET imaging detection and process device 5, an excitation fluorescence imaging and PET dual-mode imaging system is obtained. In the FMT/PET dual-mode imaging system space is arranged for the fluorescence excitation-detection device 3 and PET imaging detection and process device 5. The space around the animal stage is divided into four equal areas by two orthogonal axes on a rotation stage center. Each area is a 90 degree section. Fluorescence imaging excitation module 31 and fluorescence imaging detection module 32 employ two opposite 1/4 areas of (a) and (c), the PET imaging and detection devices 5 employ the other two 1/4 detection areas of (b) and (d).

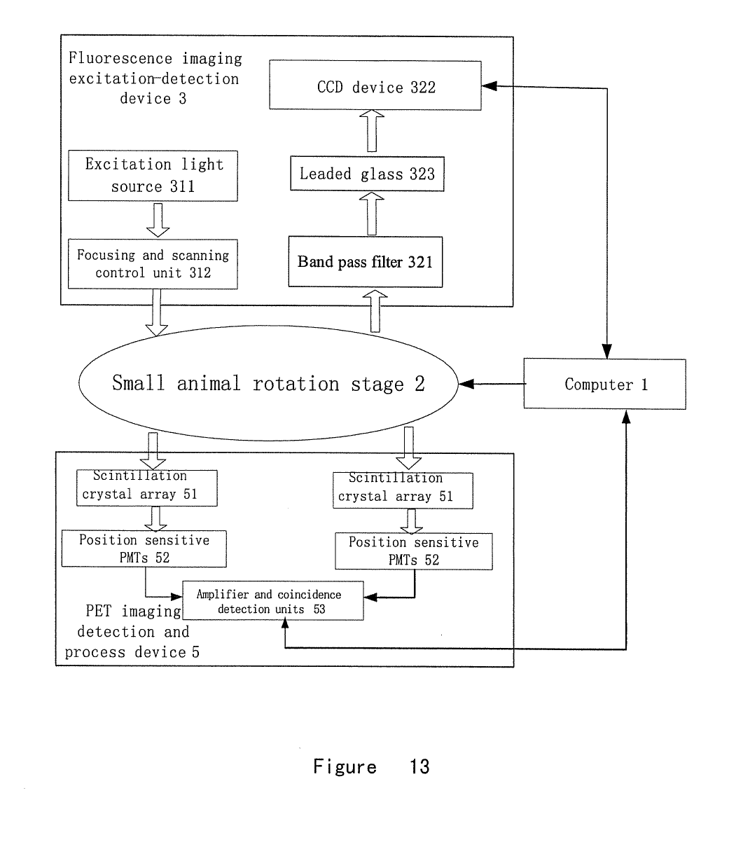

FIG. 12 illustrates the components of PET/FMT dual-modality system.

FIG. 13 is a schematic diagram of imaging data acquisition process in PET/FMT dual-modality system

FIG. 12 illustrates the components of PET/FMT dual-modality system. FIG. 13 is a schematic diagram of imaging data acquisition process in PET/FMT dual-modality system. The wide arrows indicate optical signal flows and the thick solid line arrows indicate signal flows. The PET imaging detection and process device 5 detects the gamma-rays generated by radionuclide injected into the animal body. The radionuclide is used to label specific tissues and cells. (The gamma-rays are generated by the decay of the radionuclide electronics and electronic organs.) The PET imaging detection and process device 5 includes a scintillation crystal array 51, position sensitive photomultiplier tubes 52, and amplifier and coincidence detection units 53. To prevent gamma-photon damage to CCD device 322, a piece of leaded glass 323 is placed in front of the fluorescence detection module 32. The output of the PET imaging detection and process device 5 is transferred to a digital signal to the computer 1 via an A/D conversion insert card. In the PET imaging detection and process device 5, the gamma photon is first converted to a visible light via the optical scintillation crystal material, and then is converted to an electrical signal via the position sensitive photomultiplier tubes 52. In the amplifier and coincidence detection units 53, the signal is first amplified, and then transmitted to a coincidence circuitry for processing, calculating time difference between two timing pulses arising from a coincidence event. After conditioning, the signal is finally sent to the computer 1 via an AD conversion. The output of the amplifier and coincidence detection units 5 in the PET imaging detection and process device 5 and the output of CCD device 322 in the fluorescence imaging system are sent to the computer 1. The components controlled by the computer 1 are the amplifier and coincidence detection units 53, CCD device 322, and stepper motors. In this implementation, the PET imaging detection module consists of a LYSO (lutetium-yttrium oxyorthosilicate, silicate Lutetium: yttrium) scintillation crystals in dimension of 1.9 mm×1.9 mm×10 mm, and the position sensitive photomultiplier tubes R8520 manufactured by Hamamatsu.

This invention is a dual-modality imaging system for small animal optical and positron emission tomography imaging, which is capable of multi-mode data processing, image reconstruction, image registration, and 3D visualization. The computer software controls the imaging modalities to achieve signal acquisition, data analysis, and image reconstruction.

The invention is an expandable imaging system for a small animal with the features of spatial arrangement, uniform rotation stage and continuous data acquisition. If the PET imaging detection and process device 5 is replaced by a X-ray emission and detection devices, it is capable of obtaining X-CT images of small animal simultaneously after image reconstruction. If the 360 degree space around the animal stage is divided into six equal areas, X-ray emission and detection device, fluorescence excitation-detection imaging device and PET imaging detection and process device, each occupies two opposite 60 degree measurement areas, a multimodality imaging system integrating XCT, FMT, and PET can be obtained for animal in vivo imaging. It brings together molecular contrast related functional information with precise anatomical information of in vivo small animal.

The present invention, integrated with other related equipments, will become a universal detection technique platform for in vivo small animal molecular imaging research, medical applications, and drug development. Based on the platform, research works, such as physiological data process, image reconstruction, image fusion, and 3D visualization can be done. It will be applied to tumor localization, metastasis of cancer cells, and the further applications to basic research and drug discovery.

While this invention has been described in terms of certain preferred embodiments, the invention is not limited to such embodiments. A person of ordinary skill in the art will appreciate that numerous variations and combinations of the features set forth above can be utilized without departing from the present invention as set forth in the claims. Thus, the scope of the invention should not be limited by the preceding description but should be ascertained by reference to claims that follow.

What is claimed is:

1. A dynamic sampling imaging system for in vivo fluorescence molecular imaging of a biological sample, comprising a biological sample and a fluorescence excitation-detection apparatus, wherein said sampling imaging system is configured such that said fluorescence excitation-detection apparatus detects photons emitted from said biological sample while said biological sample rotates around an axis.

2. The dynamic sampling imaging system of claim 1, further comprising a rotatable stage for supporting said biological sample such that said biological sample rotates with said rotatable stage.

3. The dynamic sampling imaging system of claim 2, further comprising a first motor for driving said stage to rotate.

4. The dynamic sampling imaging system of claim 3, wherein said rotatable stage is movable along said axis.

5. The dynamic sampling imaging system of claim 4, further comprising a second motor for driving said rotatable stage to move along said axis.

6. The dynamic sampling imaging system of claim 5, wherein said motors are stepper motors.

7. The dynamic sampling imaging system of claim 6, further comprising a computer for controlling said motors.

8. The dynamic sampling imaging system of claim 2, wherein said fluorescence excitation-detection apparatus comprises a fluorescence excitation module and a fluorescence detection module.

9. The dynamic sampling imaging system of claim 8, wherein said fluorescence excitation module comprises a fluorescence excitation source, a focalizing lens and a scan control unit, said fluorescence detection module comprises a band-pass filter and a CCD device.

10. The dynamic sampling imaging system of claim 9, wherein said CCD device is controlled by a computer.

11. The dynamic sampling imaging system of claim 8, further comprising a biological sample contour acquisition device placed at the same side of said fluorescence excitation module.

12. The dynamic sampling imaging system of claim 11, wherein a space around said stage is divided into a first pair and a second pair of opposite areas, wherein said fluorescence excitation module and said fluorescence detection module are placed in said first pair of opposite areas, and PET detectors are placed in said second pair of opposite areas, whereby realizing dual-modality imaging.

13. The dynamic sampling imaging system of claim 11, wherein a space around said stage is divided into a first pair and a second pair of opposite areas, wherein said fluorescence excitation module and said fluorescence detection module are placed in said first pair of opposite areas, and an X-ray emission apparatus and an X-ray detection apparatus, whereby realizing dual-modality imaging.

14. The dynamic sampling imaging system of claim 11, wherein a space around said stage is divided into a first pair, a second pair and a third pair of opposite areas, wherein said fluorescence excitation module and said fluorescence detection module are placed in said first pair of opposite areas, PECT detectors are placed in the second pair of opposite areas, and an X-ray emission apparatus and an X-ray detection apparatus are placed in the third pair of opposite areas, whereby realizing tri-modality imaging.

15. The dynamic sampling imaging system of claim 1, wherein said biological sample is a small animal.

16. A dynamic sampling imaging method for in vivo fluorescence molecular imaging of a biological sample, comprising detecting photons emitted from said biological sample while rotating said biological sample.

17. The dynamic sampling imaging method of claim 16, further comprising fixing said biological sample on a rotatable stage, and rotating said stage about an axis at a uniform speed under control of a computer.

18. The dynamic sampling imaging method of claim 17, further comprising lifting or lowering said stage along said axis after said stage rotates for 360 degree.

19. The dynamic sampling imaging method of claim 18, further comprising calibrating raw image data before image reconstruction, wherein:
a calibrated excitation light spot for an acquired image is set as a center point of a curve that traces light spots on a surface of said biological sample during an exposure time; and
said raw image data outside a 90 degree field of view from said axis point is discarded, and said raw image data inside said 90 degree field of view are reserved for image reconstruction.

20. The dynamic sampling imaging method of claim 19, wherein a process of data acquisition comprising following:
focusing an excitation light of a fluorescence excitation module on said surface of said biological sample via a lens; and
capturing, by a fluorescence detection module, fluorescence photons emitted out from said surface of said biological sample.
